(19) United States
(12) Patent Application Publication
(10) Pub. No.: US 2010/0317020 A1
(43) Pub. Date: Dec. 16, 2010

(54) METHODS AND COMPOSITIONS FOR DETECTING MICROORGANISMS

(76) Inventors: Stephen B. Roscoe, Saint Paul, MN (US); Stephanie J. Moeller, Saint Paul, MN (US); Manjiri T. Kshirsagar, Saint Paul, MN (US)

Correspondence Address: 3M INNOVATIVE PROPERTIES COMPANY, PO BOX 33427, ST. PAUL, MN, 55133-3427, US

(21) Appl. No.: 12/867,091

(22) PCT Filed: Feb. 12, 2009
(86) PCT No.: PCT/US09/33847

§ 371 Date: Aug. 11, 2010

Related U.S. Application Data
(62) Division of application No. 61/028,898, filed on Feb. 14, 2008.

(51) Int. Cl. C12Q 1/68; G01N 33/569; G01N 33/53

(52) U.S. Cl. 435/6; 435/7.31; 435/7.2

(57) ABSTRACT

Methods of analyzing a sample for target microorganisms of interest are described. In particular, the methods are useful for detecting a variety of yeast and mold microorganisms based on the presence of a ubiquitous cell wall component, such as zymosan. Methods of analyzing a sample for fungal microorganisms using liposomes and/or culture media are also described.

### CROSS REFERENCE TO RELATED APPLICATIONS

This application claims the benefit of U.S. Provisional Application Ser. No. 61/028,898, filed Feb. 14, 2008, which is incorporated by reference herein.

### BACKGROUND

Fungal microorganisms (e.g., yeast and mold) are ubiquitous in nature. Although they can be useful in some areas such as antibiotic or enzyme production, fungal microorganisms (e.g., yeast and mold) are known to cause food spoilage and certain species are known to produce toxins or to cause human or animal infections.

Fungal microorganisms include a very large and diverse group of chemoheterotrophic species. There are approximately 1500 species of yeast. Most yeast species exist as unicellular organisms and reproduce by budding. Some species exist in multicellular forms and others reproduce by binary fission. Molds include the filamentous fungi which can form long multicellular hyphae. Molds reproduce by forming small spores, which are typically resistant to a number of adverse environmental conditions such as extreme temperatures.

The presence of fungal microorganisms is typically detected by standard culture techniques. The samples are placed into nutrient media and incubated for a period of time to allow for the propagation of the microorganisms. After incubation, colonies may be visible or, alternatively, the sample can be viewed microscopically to observe and identify the microorganisms. Growth-based culture tests remain the most common methods for the detection and enumeration of fungal microorganisms.

Fungal microorganisms grow at various rates. Certain yeast species can undergo cell division about every 30-40 minutes. These yeast organisms can be detected and enumerated in about 1-2 days. In contrast, certain molds require several hours for each cell division. These molds can be detected and enumerated after 4-5 days.

In spite of a number of methods currently available for detecting and enumerating fungal microorganisms, a need exists for faster, more sensitive methods to detect and enumerate fungal microorganisms.

### SUMMARY

The present disclosure relates to the detection and, optionally, enumeration of yeast and/or mold microorganisms in a sample. Notwithstanding the genetic and biochemical diversity of fungal microorganisms, the inventive methods and compositions provide for simple and simultaneous detection of a variety of yeast and mold microorganisms in a sample.

In one aspect, the present disclosure provides a method for detecting a target microorganism or component thereof. The method comprises providing a recognition element and a sample suspected of containing a target microorganism, wherein the recognition element selectively binds to a zymosan. The method further comprises providing contact between the sample and the recognition element and detecting the target microorganism. Optionally, the method may further comprise the steps of providing a nutrient medium in a culture device and incubating the sample under conditions to allow for at least one cell division.

In another aspect, the present disclosure provides a method for detecting a target microorganism or a component thereof. The method can comprise providing a recognition element wherein the recognition element selectively binds to a zymosan, a signaling element which generates a detectable signal, and a sample suspected of containing a target microorganism. The signaling element can comprise a linking moiety. The method further can comprise providing contact between the sample and the recognition element and the signaling element and detecting the detectable signal. Optionally, the method may further comprise the steps of providing a nutrient medium in a culture device and incubating the sample under conditions to allow for at least one cell division.

In another aspect, the present disclosure provides a method for detecting a target microorganism or a component thereof. The method can comprise providing a recognition element, a signaling element which generates a detectable signal, and a sample suspected of containing a target microorganism. The recognition element can be linked to the signaling element and can selectively bind to a zymosan. The method further can comprise providing contact between the sample and the recognition element and detecting the detectable signal. Optionally, the method may further comprise the steps of providing a nutrient medium in a culture device and incubating the sample under conditions to allow for at least one cell division.

In another aspect, the present disclosure provides a method for detecting a target microorganism or a component thereof. The method can comprise providing a lipid vesicle comprising a lipid bilayer and a signaling element which generates a detectable signal, a recognition element which selectively binds to a cell wall component, and a sample suspected of containing the target microorganism. The method further can comprise providing contact between the sample, the recognition element, and the lipid vesicle under conditions effective to cause the binding of the lipid vesicle to the target microorganism, if one is present, and detecting the detectable signal. Optionally, the method may further comprise the steps of providing a nutrient medium in a culture device and incubating the sample under conditions to allow for at least one cell division.

In another aspect, the present disclosure provides a composition for detecting a target microorganism. The composition can comprise a capture agent, which can bind selectively to a zymosan, and a signaling element which generates a detectable signal.

The terms “analyte” and “antigen” are used interchangeably and refer to various molecules (e.g., zymosan) or epitopes of molecules (e.g., different binding sites of zymosan), or whole cells of the microorganism, that are characteristic of a microorganism (i.e., microbe) of interest. These include components of cell walls (e.g., cell-wall proteins), external cell wall components (e.g., mannan, chitin, or zymosan), internal cell components (e.g., cytoplasmic membrane proteins), etc.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, a sample suspected of containing “a” target microorganism can be interpreted to mean that the sample can include “one or more” target microorganisms.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawing figures listed below, where like structure is referenced by like numerals throughout the several views.

FIG. 1 is a representation of the binding interaction between a signaling element and a target microorganism according to one embodiment of the present invention.

FIG. 2a is a representation of the binding interactions between target microorganism, a recognition element, and a labeled secondary recognition element according to one embodiment of the present invention.

FIG. 2b is a representation of the binding interactions between a target microorganism, a biotin-labeled recognition element and a streptavidin-labeled signaling element according to one embodiment of the present invention.

FIG. 3 is a representation of the binding interactions between a capture agent attached to a support material, a cell wall component, a biotin-labeled recognition element, and a streptaidin-labeled signaling element, according to one embodiment of the present invention.

FIG. 4a is a representation of the binding interactions between a target microorganism, a biotin-labeled recognition element, and a biotin-labeled lipid vesicle containing a latent signaling element, according to one embodiment of the present invention.

FIG. 4b is a representation of the activation of the latent signaling element of FIG. 4a.

FIG. 5a is a representation of the binding interactions of a target microorganism, a biotin-labeled recognition element, streptavidin, and a biotin-labeled lipid vesicle containing a latent signaling element, according to one embodiment of the present invention.

FIG. 5b is a representation of the activation of the latent signaling element of FIG. 5a.

FIG. 6a is a representation of the binding interactions of a capture agent attached to a support material, a cell wall component, a biotin-labeled recognition element, streptavidin, and a biotin-labeled lipid vesicle containing a latent signaling element, according to one embodiment of the present invention.

FIG. 6b is a representation of the activation of the latent signaling element of FIG. 6a.

FIG. 7a is a block diagram of a method using a capture agent for detecting a target microorganism in a sample.

FIG. 7b is a block diagram of a method for detecting a target microorganism in a sample.

FIG. 7c is a block diagram of a method using a growth step for the detection of a target microorganism in a sample.

### DETAILED DESCRIPTION

The diversity of yeast and mold microorganisms presents a challenge when trying to develop a rapid method for the detection of most or all species within those groups. The diversity can be seen by the differences in the genomes and proteomes of the fungal microorganisms. The diversity is also reflected by the relatively broad range of growth rates of the microorganisms on traditional growth media.

The present disclosure relates to methods and compositions for rapidly detecting a wide variety of yeast and mold microorganisms. The methods include the use of a recognition element, which selectively binds to an analyte (e.g., cell wall component) present in a large number of diverse fungal microorganisms, to deliver a signaling element for detecting the microorganisms. Zymosan, a glucan found in the cell wall of a number of fungal microorganisms, is a preferred analyte for the detection of yeast and mold microorganisms.

The major polysaccharides of the fungi cell wall matrix consist of noncellulosic glucans such as zymosan and glycogen-like compounds, mannans (polymers of mannose), chitosan (polymers of glucosamine), and galactans (polymers of galactose). Small amounts of fucose, rhamnose, xylose, and uronic acids may be present.

Many fungi, especially the yeasts, have soluble peptidomannans as a component of their outer cell wall in a matrix of α- and β-glucans. Mannans, galactomannans, and, less frequently, rhamnomannans are responsible for the immunologic response to many medically important yeasts and molds. Mannans are polymers of mannose or heteroglucans with α-D-mannan backbones. Structurally, mannan consists of an inner core, outer chain, and base-labile oligomannosides.

Signaling elements used to detect the microorganisms fall into two major classes-manifest signaling elements and latent signaling elements. Manifest signaling elements are molecules or compositions which can be detected readily by at least one of a number of means such as fluorescence. Nonlimiting examples of manifest signaling elements include dyes (e.g., fluorescent dyes), particles (e.g., magnetic, polymeric, or gold particles, which may be optionally labeled with a dye), polypeptides (e.g., enzymes or fluorescent proteins such as GFP or YFP), and other chemical groups (e.g. polyoxyalkylenes) which can be labeled with a detectable moiety (e.g., fluorescein) and be attached to a recognition element (e.g., an antibody). Attachment of signaling elements to the recognition element preferably does not interfere with the interaction between the recognition element and its corresponding binding partner.

Latent signaling elements are molecules or compositions which are sequestered or shielded, such that they cannot be detected readily until the sequestering or shielding is reduced to an extent that the signaling element becomes detectable. Nonlimiting examples of latent signaling elements include enzymes, enzyme substrates, dyes, and/or fluorescent molecules sequestered within a lipid vesicle. The sequestration of an enzyme from its corresponding substrate prevents a chromogenic or fluorogenic reaction. Fluorescent molecules may be packaged at high enough concentrations in a lipid vesicle to promote fluorescence quenching. Alternatively, the fluorescent molecules may be packaged in a lipid vesicle with a corresponding quenching reagent. Upon disruption of the lipid vesicles, the concentration of the fluorescent molecules and/or quenching reagents may become dilute enough to overcome the quenching effect and thereby become detectably fluorescent.

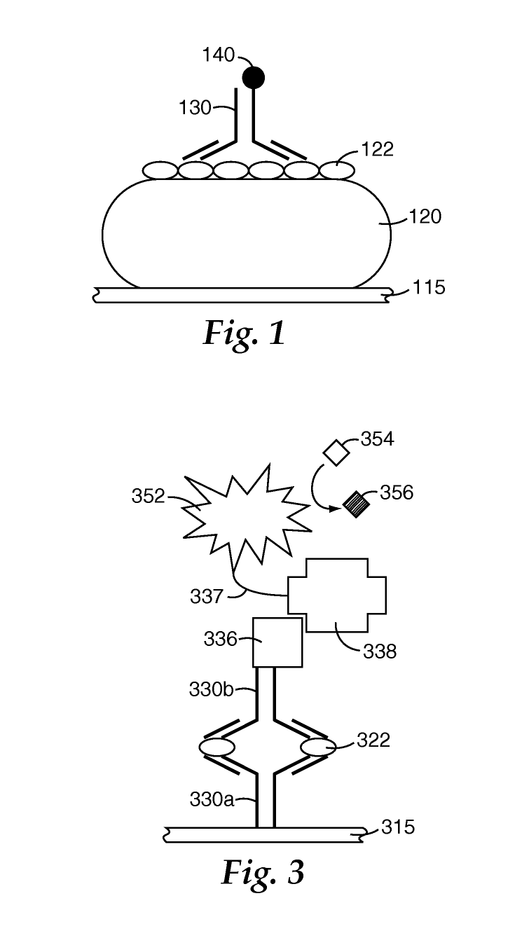

FIG. 1 shows one embodiment of a method for the detection of a target microorganism using a manifest signaling element which is directly attached to a recognition element. In this embodiment, a sample containing a target microorganism 120 comprising an analyte 122 (e.g., zymosan) is mixed with a recognition element 130 (e.g., an antibody) which selectively binds to the analyte 122. The recognition element 130 further comprises a signaling element 140 (e.g., a fluorescent dye), which is attached directly to the recognition element 130. Various methods to attach small molecules (e.g. biotin or fluorescent dyes) or polypeptides (e.g. enzymes) to antibodies are known in the art (see, for example, “Antibodies, A Laboratory Manual”; E, Harlow and D. Lane, eds.; 1988; Cold Spring Harbor Laboratory Press; Cold Spring harbor, NY). Fluorescein isothiocyanate (FITC) is an example of a fluorescent signaling element which can be attached directly to a recognition element.

Also shown in FIG. 1 is an optional support material 115. In the inventive method, the target microorganism 120 can be freely suspended in a liquid phase or, alternatively, can be attached to a support material 115. Nonlimiting examples of support materials 115 include plastic films, particles, or substrates (e.g., microtiter plates); glass films, particles or substrates (e.g., glass beads or glass slides); metal films, particles, or substrates; membranes and/or filters (e.g. glass fiber filter, nylon or cellulose nitrate); ceramic particles or substrates; hydrogels (e.g., agarose or polyacrylamide), or combinations of any two or more of the foregoing. A number of methods of fixing target microorganisms 120 to support materials 115 are known in the art. Nonlimiting examples of fixing target microorganisms 120 include heat fixing, chemical cross-linking (e.g., using glutaraldehyde), antibody capture, and the like. The method of fixing the target microorganism 120 should be selected such that it does not mask, alter, or destroy all of the analyte 122, such that the recognition element 130 is prevented from selectively binding to the analyte 122.

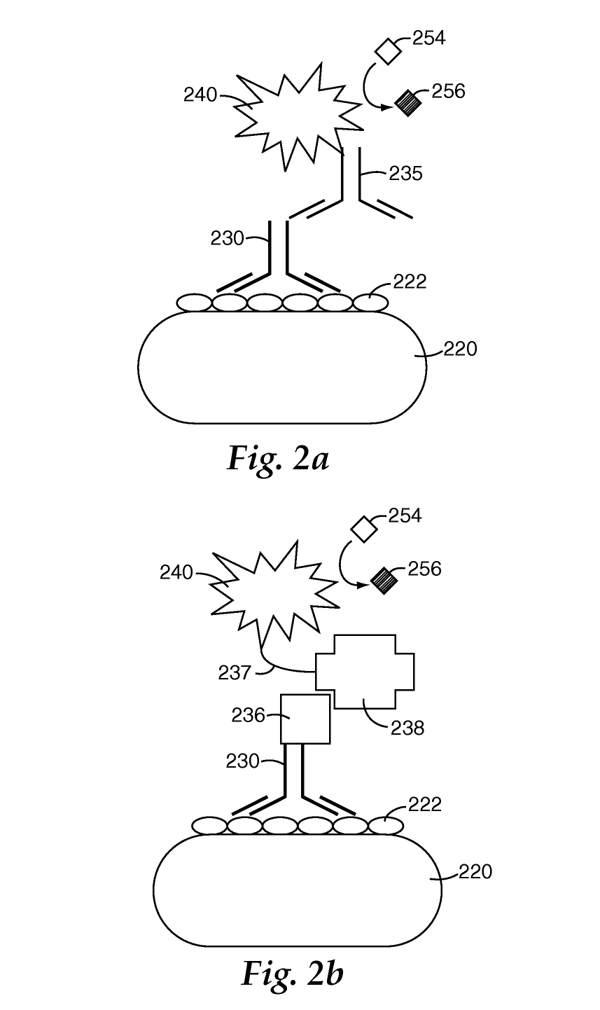

FIG. 2a shows an embodiment of a method for the detection of a target microorganism using a manifest signaling element which is indirectly attached to a recognition element. In this embodiment, a sample containing a target microorganism 220 comprising an analyte 222 (e.g., zymosan) is mixed with a recognition element 230 (e.g., an antibody) which selectively binds to the analyte 222. A secondary recognition element 235 (e.g., a secondary antibody), labeled with a signaling element 240, selectively binds to the recognition element 230. In this embodiment, signaling element 240 comprises an enzyme activity which converts enzyme substrate 254 into a detectable enzyme product 256. The enzyme reaction may be detectable by a number of means known in the art such as, for example, a color change (e.g. light absorbance or reflectance), by fluorescence, by impedance or conductance, or by luminescence. As described above, the target microorganism 220 can be freely suspended in a liquid phase or, alternatively, can be attached to a support material (not shown).

FIG. 2b shows an alternative embodiment of a method for the detection of a target microorganism using a manifest signaling element which is indirectly attached to a recognition element. In this embodiment, a sample containing a target microorganism 220 comprising an analyte 222 (e.g., zymosan) is mixed with a recognition element 230 (e.g., an antibody) which selectively binds to the analyte 222 and which comprises a biotin group 236. A biotin-binding molecule 238 (e.g., avidin or streptavidin), labeled with a signaling element 240, selectively binds to the biotin group 236. In this embodiment, signaling element 240 comprises an enzyme activity which converts enzyme substrate 254 into a detectable enzyme product 256. Signaling element 240 is attached to biotin-binding molecule 238 via linker 237. Linkers 237, which are used to join various molecules, such as two polypeptides, are known in the art and are described in more detail below. The enzyme reaction may be detectable by a number of means as described above. As described above, the target microorganism 220 can be freely suspended in a liquid phase or, alternatively, can be attached to a support material (not shown).

FIG. 3 shows an embodiment of a method for the detection of a target microorganism using a manifest signaling element in a sandwich-type assay. In this embodiment, a capture agent 330a (e.g., an antibody or receptor which selectively binds to zymosan) is attached to support material 315. A sample containing an analyte 322 (e.g., zymosan) is contacted with a recognition element 330b (e.g., an antibody or receptor which selectively binds to zymosan) labeled with biotin 336, a biotin-binding molecule 338 (e.g., avidin or streptavidin) labeled with signaling element 352, and the capture agent 330a (attached to support material 315), to form the complex illustrated in FIG. 3. In this embodiment, signaling element 352 comprises an enzyme activity which converts enzyme substrate 354 into a detectable enzyme product 356. In this and other embodiments, signaling element 352 can be attached to biotin-binding molecule 338 via an optional linker 337, provided the linker 337 does not prevent the biotin-binding molecule 338 from binding to biotin 336 and does not block the enzyme activity of signaling element 352. Examples of linkers 337 are described below. Support material 315 can be constructed in a number of forms (e.g., films, membranes, particles) and from a number of different materials. Nonlimiting examples of support materials 115 are described above.

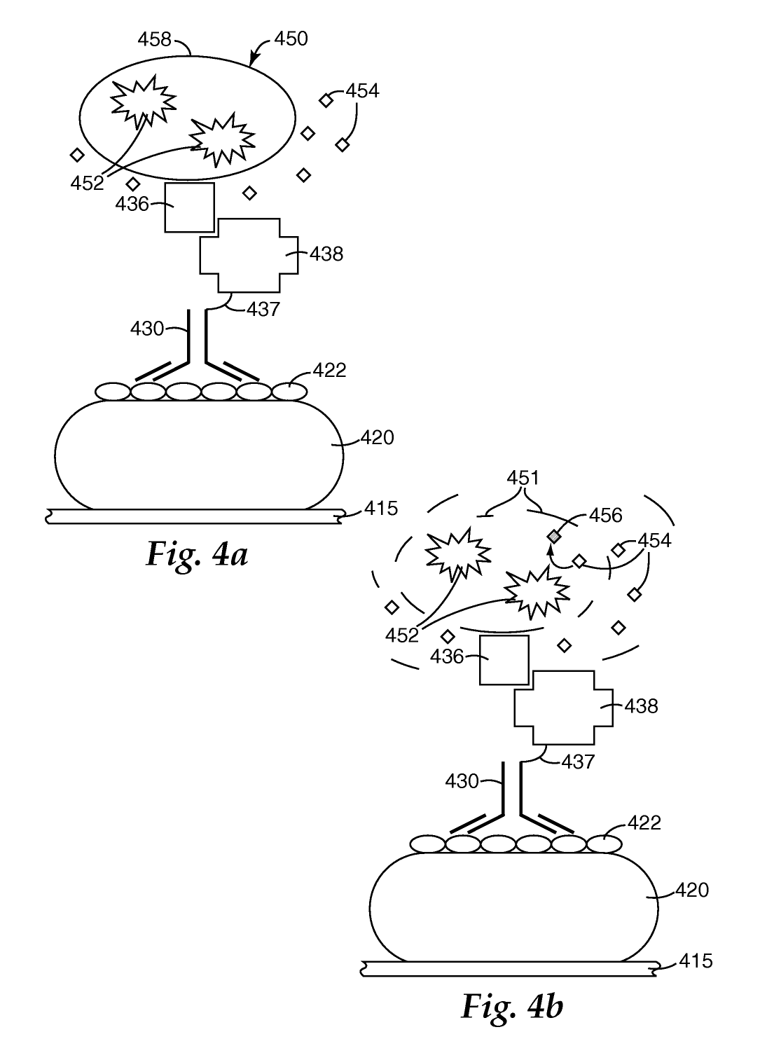

FIG. 4a shows an embodiment of a method for the detection of a target microorganism using a latent signaling element. In this embodiment, a sample containing a target microorganism 420 comprising an analyte 422 (e.g., zymosan) is mixed with a recognition element 430 (e.g., an antibody or a composition comprising the carbohydrate-recognition domain from Dectin-1), which selectively binds to the analyte 422, and a liposome 450. In this embodiment, the liposome 450 functions as a latent signaling element because the interaction between the liposome 450, the recognition element 430, and the target microorganism 420 per se does not necessarily result in a detectable signal. Rather, in this embodiment, an additional step (shown in FIG. 4b) can produce the detectable signal. In this embodiment, the recognition element 430 further comprises a biotin-binding molecule 438 (e.g. avidin or streptavidin) which is attached to the recognition element via an optional linker 437. Alternatively, the biotin-binding element 438 can be attached directly to the recognition element 430.

The liposome 450 comprises an enzyme 452 and biotin 436, which is selectively bound by the biotin-binding element 438. The liposome 450 may be constructed from phospholipids comprising biotin 436. For example, the bilayer membrane 458 may be synthesized from N-Biotinyl-1,2-Dipalmitoyl-sn-Glycero-3-Phosphoethanolamine (Avanti Polar Lipids, Alabaster, Ala.), as described in Example 3, and/or synthesized from nonbiotinylated phospholipids. The enzyme 452 may be present (e.g. in an aqueous suspension) within the liposome 450 (as shown in FIG. 4a). Alternatively, the enzyme 452 may be physically associated with the bilayer membrane 458 of the liposome 450. As used herein, “physically associated” means that the enzyme may span both lipid layers of the bilayer membrane 458 (e.g., with at least a portion of the enzyme on both sides of the bilayer membrane 458) or that the enzyme 452 may partition into the inner or outer lipid layer of the bilayer membrane 458. In some embodiments, the enzyme 452 may be modified with chemical adducts (e.g., alkyl adducts) to promote physical association with the bilayer membrane 458.

FIG. 4b shows how the latent signaling element (enzyme 452) of FIG. 4a can be activated to produce a detectable signal. In this embodiment, the bilayer membrane 458 of the liposome 450 is disrupted, thus allowing the enzyme 452 to contact the enzyme substrate 454 and convert it to product 456. The conversion of the enzyme substrate 454 to product 456 can result in the generation of a detectable signal. The detectable signal may be detectable optically. Nonlimiting means of optical detection include colorimetric detection, fluorometric detection, or lumimetric (e.g. bioluminescence or chemiluminescence) detection. The detectable signal may be observed visually or, alternatively, may be detected with an instrument such as a spectrophotometer, a fluorimeter, or a luminometer, for example. Alternatively, the signal may be detected by a change in impedance or conductance in the mixture or by a change in the pH of the mixture.

The permeability of the bilayer membrane 458 may be disrupted by a number of means, which may result in the formation of liposome fragments 451. Nonlimiting examples of disruption means include sonic vibration, a freeze-thaw process, heating, chemical (e.g. a surfactant) disruption, osmotic disruption (e.g. osmolysis), or by permeabilization with a pore-forming agent such as a cytolytic peptide (described below). Disrupting the permeability of the bilayer membrane 458 may permit the passage of the enzyme 452 out of the liposome 450, passage of the enzyme substrate 454 into the liposome 450, or both. Also shown in FIG. 4b is linker 437, which joins recognition element 430 to biotin-binding molecule 438.

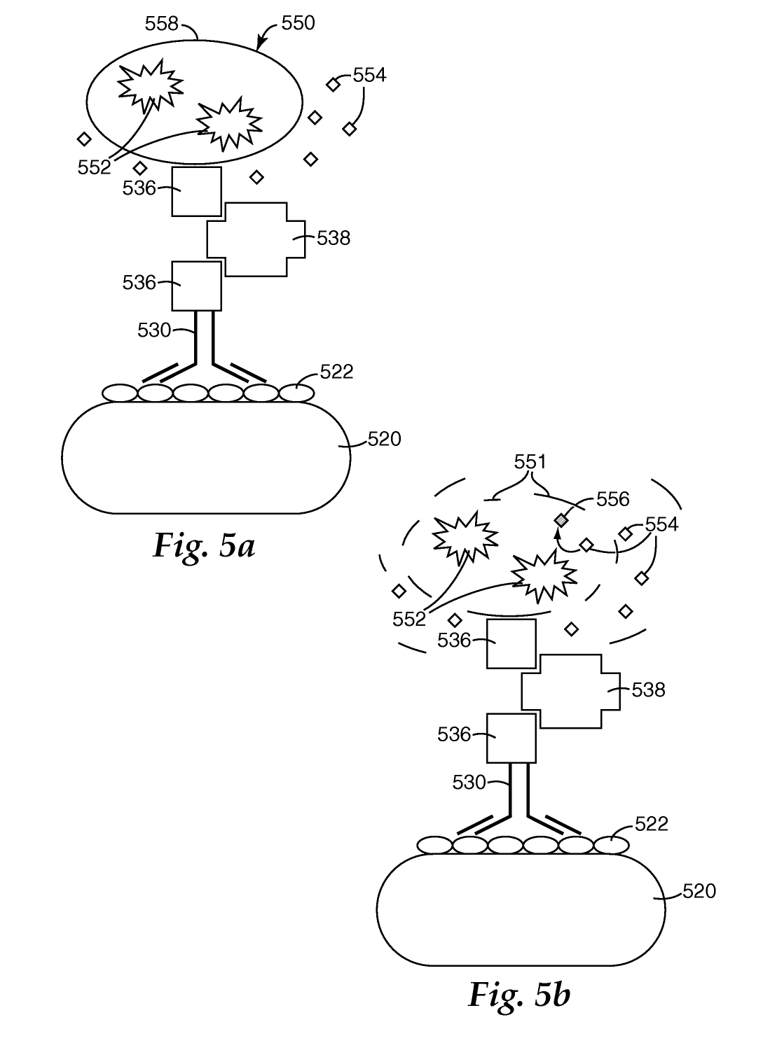

FIG. 5a shows an alternative embodiment of a method for the detection of a target microorganism using a latent signaling element. In this embodiment, a sample containing a target microorganism 520 comprising an analyte 522 (e.g., zymosan) is mixed with a recognition element 530 (e.g., an antibody or a composition comprising the carbohydrate-recognition domain from Dectin-1) labeled with biotin 536, which selectively binds to the analyte 522, a biotin-binding molecule 538, and a liposome 550 comprising biotin 536. The biotin-binding molecule 538 can form a bridge between the recognition element 530 and the liposome 550. In this embodiment, the liposome 550 functions as a latent signaling element because the interaction between the liposome 550, the recognition element 530, and the target microorganism 520 per se does not necessarily result in a detectable signal. Rather, in this embodiment, an additional step (shown in FIG. 5b) can produce the detectable signal. In this embodiment, the recognition element 530 further comprises biotin 536. Also shown in FIG. 5a is an enzyme substrate 554.

The liposome 550 comprises an enzyme 552 and biotin 536, which is selectively bound by the biotin-binding molecule 538. The liposome 550 may be constructed from phospholipids comprising biotin 536. For example, the bilayer membrane 558 may be constructed from N-Biotinyl-1,2-Dipalmitoyl-sn-Glycero-3-Phosphoethanolamine (Avanti Polar Lipids, Alabaster, Ala.), as described in Example 3, and/or synthesized from nonbiotinylated phospholipids. The enzyme 552 may be present (e.g. in an aqueous suspension) within the liposome 550 (as shown in FIG. 5a). Alternatively, the enzyme 552 may be physically associated with the bilayer membrane 558 of the liposome 550. In some embodiments, the enzyme 552 may be modified with chemical adducts (e.g., alkyl adducts) to promote physical association with the bilayer membrane 558.

FIG. 5b shows how the latent signaling element (enzyme 552) of FIG. 5a can be activated to produce a detectable signal. In this embodiment, the bilayer membrane 558 of the liposome 550 is disrupted, thus allowing access for the enzyme 552 to contact the enzyme substrate 554 and convert it to product 556. The conversion of the enzyme substrate 554 to product 556 can result in the generation of a detectable signal. The detectable signal may be detectable optically as described above or alternatively, the signal may be detected by a change in impedance or conductance in the mixture or by a change in the pH of the mixture. The permeability of the bilayer membrane 558 may be disrupted by a number of means, which may result in the formation of liposome fragments 551, as described above. Disrupting the permeability of the bilayer membrane 558 may permit the passage of the enzyme 552 out of the liposome 550, passage of the enzyme substrate 554 into the liposome 550, or both.

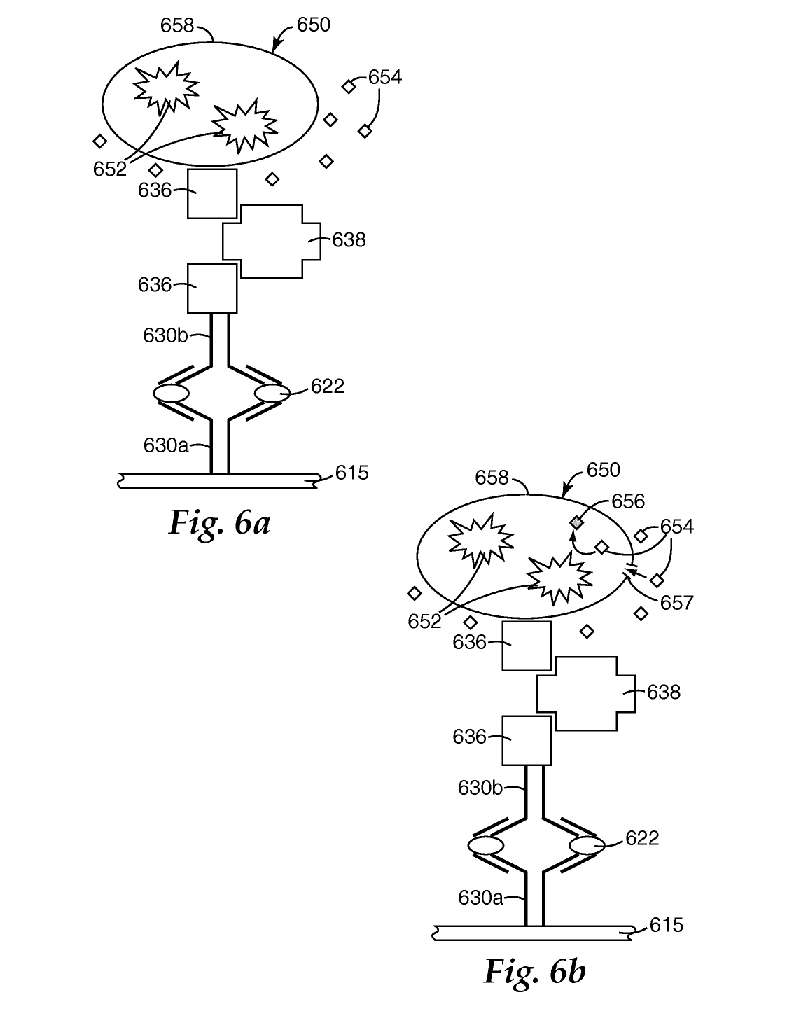

FIG. 6a shows an embodiment of a method for the detection of a target microorganism using a latent signaling element in a sandwich-type assay. In this embodiment, a capture agent 630a (e.g., an antibody which selectively binds to zymosan) is attached to support material 615. A sample containing an analyte 622 (e.g., zymosan) is contacted with a recognition element 630b (e.g., an antibody or receptor which selectively binds to zymosan) labeled with biotin 636, a biotin-binding molecule 638 (e.g., avidin or streptavidin), a liposome comprising biotin 636, and the capture agent 630a (attached to support material 615), forming the complex illustrated in FIG. 6a. Alternatively, the analyte 622 may be attached to a target microorganism or a fragment thereof (not shown). Liposome 650 further comprises an enzyme 652. In this embodiment, the liposome 650 functions as a latent signaling element because the interaction between the liposome 650, the recognition elements 630a and 630b, and the analyte 622 per se does not necessarily result in a detectable signal. Rather, in this embodiment, an additional step (shown in FIG. 6b) can allow the enzyme 652 to contact enzyme substrate 654 and, thereby, produce the detectable signal.

The liposome 658 comprises an enzyme 652 and biotin 636, which is selectively bound by the biotin-binding molecule 638. The liposome 658 may be constructed from phospholipids comprising biotin 636. For example, the bilayer membrane 658 may be constructed from N-Biotinyl-1,2-Dipalmitoyl-sn-Glycero-3-Phosphoethanolamine (Avanti Polar Lipids, Alabaster, Ala.) as described in Example 3. The enzyme 652 may be present (e.g. in an aqueous suspension) within the liposome 650 (as shown in FIG. 5a). Alternatively, the enzyme 652 may be physically associated with the bilayer membrane 658 of the liposome 650. In some embodiments, the enzyme 652 may be modified with chemical adducts (e.g., alkyl adducts) to promote physical association with the bilayer membrane 658.

FIG. 6b shows how the latent signaling element (enzyme 652) of FIG. 6a can be activated to produce a detectable signal. In this embodiment, the bilayer membrane 658 of the liposome 650 is disrupted by a signal-generating element such as a pore-forming polypeptide (not shown), thus allowing the enzyme substrate 654 to enter the liposome through pore 657 and contact the enzyme 652. Alternatively, the liposome 650 may be disrupted by other chemical and/or mechanical means described above.

A list of exemplary polypeptides which can permeabilize liposomes is shown in Table 1. The lysis of liposome particles by polypeptides is disclosed in International Patent Application No. PCT/GB98/03071, entitled “PARTICLES”, and in International Patent Application Number PCT/GB02/00033, entitled “ARMED PEPTIDES”, which herein are incorporated by reference in their entirety. After the permeabilization of bilayer membrane 658, enzyme 652 converts enzyme substrate 654 to product 656. The conversion of the enzyme substrate 654 to product 656 can result in the generation of a detectable signal, as described above. Disrupting the permeability of the bilayer membrane 658 may permit the passage of the enzyme 652 out of the liposome 650, passage of the enzyme substrate 654 into the liposome 650, or both. Support material 615 can be constructed as described above.

TABLE 1
Peptides which can disrupt the permeability of liposomes.

A-23187
(Calcium ionophore)
Aerolysin
Amphotericin B
Ascaphin
Aspergillus
haemolysin
Alamethicin
Apolipoproteins
ATP Translocase
Bombinin
Brevinin
Buforin
Caerin
Cereolysin
Colicins
Dermadistinctin
Dermaseptin
Dermatoxin
Direct lytic factors
from animal venoms
Diptheria toxin
Distinctin
Esculetin
Filipin
Gaegurin
GALA
Gramicidin
Helical erythrocyte
lysing peptide
Hemolysins
Ionomycin
KALA
LAGA
Listerolysin
Maculatin
Magainin
Maxymin
Melittin
Metridiolysin
Nigericin
Nystatin
Ocellatin
P25
Palustrin
Phospholipases
Phylloxin
Polyene Antibiotics
Polymyxin B
Ranalexin
Ranateurin
Rugosin
Saponin
Staphylococcus aureus
toxins (α, β, χ, δ)
Streptolysin O
Streptolysin S
Synexin
Surfactin
Tubulin
Valinomycin
Vibriolysin

### Samples

In certain embodiments, the fluid samples comprise a food or beverage. Methods for the preparation of food samples for microbiological analyses are well known. Some of the sample preparation methods for food samples involve suspending a known quantity of food material (25 grams, for example) in a relatively large volume of diluent (225 milliliters, for example). The sample is subjected to a strenuous mixing process, such as blending or stomaching, to create a relatively homogeneous liquid suspension. The samples are frequently processed in a plastic sample reservoir which is called a stomacher bag. Methods and compositions of the present disclosure provide a way to analyze food or beverage samples. Nonlimiting examples of foods which are routinely tested for microorganisms include meat (e.g., ground meat, poultry, fish, seafood), fresh or processed produce (e.g., fruit, vegetables), dairy (e.g., milk or milk products, whey, cheese), and beverages (e.g., milk, water, fruit juices, vegetable juices, tea).

In some embodiments, samples to be processed and analyzed include samples from a body of water. Nonlimiting examples of such bodies of water include surface water, water for human or animal consumption, and water used for industrial processes. Surface water includes an ocean, a lake, a river, a canal, a pond, a reservoir, a stream, and the like. Process water includes water that is used in municipal or industrial purposes, such as cleaning, washing, rinsing, cooling towers, water treatment holding tanks, and the like. Exemplary cleaning processes include food processing processes, such as, washing, rinsing, and disinfecting meat or produce for human or animal consumption.

In other embodiments, the methods and compositions of the present disclosure are used to analyze samples that are amenable to processing and microbial detection such as, for example, solutions, mixtures, homogenates, or liquid suspensions of foodstuffs, beverages and pharmaceutical products. In certain embodiments, the liquid sample comprises one or more dissolved solute, such as sugars, salts, or proteins. In other embodiments, the liquid sample may comprise one or more solvent, such as an alcohol, or a surfactant. Samples with solvents or surfactants can be used in accordance with the present invention, provided the solvents or surfactants are present at a concentration which does not prevent the detection of the detectable signal or cause the inadvertent conversion of a latent signaling element to a detectable signal (e.g., causing a detectable signal when there are no target microorganisms present in the sample). Samples which, when mixed with a pH-sensitive signaling element (e.g., a fluorescent label such as 4-methylumbelliferone) or a pH-responsive signal-generating element (e.g., pH-triggered cytolytic peptides), may be buffered and/or adjusted to a compatible pH prior to mixing with the pH-sensitive or pH-responsive elements.

In some embodiments, the methods and compositions of the present disclosure can be used to detect microorganisms in an environmental or clinical sample. Typically, environmental or clinical samples are collected using a swab, a sponge, a wipe, or the like to collect residual material from a surface (e.g., a counter top, a floor, skin, a wound site) which may be contaminated with microorganisms. The collection device can be transferred to a sample reservoir and mixed or homogenized with a solvent (e.g., Standard Methods Buffer, buffered peptone water, buffered saline, or distilled water) to release the microorganisms into the solvent. Subsequently, the solvent can be analyzed for the presence of a microorganism. Alternatively, the target microorganisms may be analyzed in a solution containing the collection device.

Individual liquid samples may contain almost any number and kind of microorganism. The number of microorganisms in a liquid sample may range from zero organisms per milliliter, in a sample that has been subjected to sterilizing conditions, up to approximately 10⁹ or more organisms per milliliter in a heavily-contaminated sample. The devices and methods of the present invention provide for the analysis of liquid samples containing a wide variety of microbial concentrations.

### Recognition Elements

The present disclosure includes the use of recognition elements that selectively bind to a cell wall component, such as zymosan, of a target microorganism. In some embodiments, recognition elements include antibodies, such as monoclonal or polyclonal antibodies. The antibodies can be obtained from a number of organisms such as mice, rabbits, goats, and the like, using methods that are well known in the art such as those described in “Antibodies, A Laboratory Manual” from Cold Spring Harbor Press, Cold Spring Harbor, N.Y.

Also included in the present invention are various antibody fragments, also referred to as antigen binding fragments, which include only a portion of an intact antibody, generally including an antigen binding site of the intact antibody and thus retaining the ability to bind antigen. Fragments can be obtained via chemical or enzymatic treatment of an intact or complete antibody or antibody chain. Fragments can also be obtained by recombinant means. Examples of antibody fragments include, for example, Fab, Fab′, Fd, Fd′, Fv, single-chain Fv, dAB, and F(ab′)₂ fragments produced by proteolytic digestion and/or reducing disulfide bridges and fragments produced from an Fab expression library. Such antibody fragments can be generated by techniques well known in the art. Antibodies of the present invention can include the variable region(s) alone or in combination with the entirety or a portion of the hinge region, CH1 domain, CH2 domain, CH3 domain and/or Fc domain(s). The term “antigen-binding fragment” refers to a polypeptide fragment of an immunoglobulin or antibody that binds antigen or competes with intact antibody (i.e., with the intact antibody from which they were derived) for antigen binding (i.e., specific binding).

Monoclonal antibodies of the present invention include, but are not limited to, humanized antibodies, chimeric antibodies, single chain antibodies, single-chain Fvs (scFv), disulfide-linked Fvs (sdFv), Fab fragments, F(ab′) fragments, F(ab′)₂ fragments, Fv fragments, diabodies, linear antibodies fragments produced by a Fab expression library, fragments including either a VL or VH domain, intracellularly-made antibodies (i.e., intrabodies), and antigen-binding antibody fragments thereof.

Monoclonal antibodies of the present invention may be of any isotype. The monoclonal antibodies of the present invention may be, for example, murine IgM, IgG1, IgG2a, IgG2b, IgG3, IgA, IgD, or IgE. The monoclonal antibodies of the present invention may be, for example, human IgM, IgG1, IgG2, IgG3, IgG4, IgA1, IgA2, IgD, or IgE. In some embodiments, the monoclonal antibody may be murine IgG2a, IgG1, or IgG3. With the present invention, a given heavy chain may be paired with a light chain of either the kappa or the lambda form.

In alternative embodiments, recognition elements include polypeptide receptors, for cell wall components. Polypeptide receptors typically comprise at least one domain which selectively binds to a specific analyte. An exemplary polypeptide receptor for zymosan is the Dectin-1 receptor. Dectin-1 comprises a carbohydrate-recognition domain. In some embodiments, recognition elements also include ligand-binding fragments of the polypeptide receptors (e.g., the carbohydrate-recognition domain of Dectin-1). The receptor may be purified from a natural source such as, in the case of Dectin-1, human monocyte cells. Alternatively, the polypeptide receptor may be produced by cloning and expressing the genetic material encoding the Dectin-1 polypeptide, or the carbohydrate-recognition domain thereof, in a suitable host organism.

The recognition elements can be modified with a chemical constituent, provided the chemical modification does not prevent the recognition element from binding to the analyte (e.g. cell wall component). The chemical constituent may be a signaling element, for example. A recognition element which binds to a predetermined structure on the target cell may be inserted into the bilayer membrane of the liposome. For example, the Dectin-1 receptor, or a derivative thereof, may be inserted into the liposome and used to bind zymosan. Derivatives can include long-chain alkyl groups (e.g., a myristic acid derivative), which can promote the association of the polypeptide with the liposome.

### Signaling Elements

Signaling elements function to provide a detectable signal which, when associated with the target microorganism, can indicate the presence of a target microorganism in a sample. Signaling elements can be attached directly (e.g., covalent coupling) to a recognition element. Alternatively, signaling elements can become associated with a recognition element by indirect attachment (e.g., the formation of a complex consisting of a biotinylated recognition element, streptavidin, and a biotinylated signal element). Signaling elements may comprise a polypeptide (e.g., a fluorescent polypeptide such as green fluorescent protein or yellow fluorescent protein or an enzyme) or a polynucleotide (e.g., a labeled polynucleotide).

Signaling elements may be manifest signaling elements, such that they can provide an immediate and/or obvious detectable signal (e.g., a fluorescent dye). Alternatively, signaling elements may be latent signaling elements, such that they must be activated in order to provide a detectable signal. Nonlimiting examples of latent signaling elements include a fluorescent dye which is quenched by, for example, fluorescence energy transfer and an enzyme which is sequestered from its corresponding enzyme substrate.

Signaling elements may be incorporated into a lipid vesicle or liposome. The signaling element incorporated within the particle may be any chosen compound. For example, the signaling element may be a relatively small molecule such as a dye or electrochemical mediator (e.g., a polyelectrolyte, which may affect electrochemcial conductivity). The signaling element may also be a fluorescent molecule (such as latex or polystyrene), an antibody, hormone or an enzyme.

### Signal-Generating Elements

A “signal-generating element”, as used herein, refers to a molecule (e.g., a polypeptide or a chemical agent) which can cause a latent signaling element to generate a detectable signal. An enzyme which is sequestered in a lipid vesicle and, as such, cannot react freely with a corresponding enzyme substrate, is an example of a latent signaling element. An exemplary signal-generating element can be a cytolytic polypeptide which can modulate the permeability of the lipid vesicle such that the enzyme substrate and/or enzyme may pass through the bilayer membrane of the lipid vesicle, thereby allowing the enzyme reaction to proceed. The lipid vesicle (comprising the signaling element) may be adapted such that the species can be activated in a number of ways. For instance, the permeability of the lipid bilayer of a lipid vesicle may be regulated such that permeability across the bilayer may be increased in response to a predetermined metabolic signal. According to the first aspect of the invention this may be achieved by incorporating peptides that act as cytolytic agents into the vesicle. Exemplary cytolytic peptides are listed in Table 1 and are described in International Patent Application No. PCT/GB98/03071, entitled “PARTICLES”, in International Patent Application Number PCT/GB02/00033, entitled “ARMED PEPTIDES”, and in U.S. Patent Application No. 61/028,896, filed Feb. 14, 2008 and entitled “POLYPEPTIDES FOR MICROBIAL DETECTION”, which herein are incorporated by reference in their entirety. The peptides may be large proteins or short polypeptides provided that they are capable of modulating permeability of the particle.

The signal-generating elements may act to open or mediate the opening of pores or channels within the lipid bilayer to allow molecules (e.g., enzyme substrates) to enter into the vesicle and thereby produce a detectable signal. Alternatively, or additionally, the opening of pores or channels within the lipid bilayer may allow the release of the substance into the extravesicular environment. The cytolytic agents may even cause the rupture of the lipid vesicle to allow the release of the species contained therein. The cytolytic agents may be activatable (e.g., responsive to a chemical or biochemical released from the cell). The cytolytic peptide may create an ion channel which “opens” in response to ions (e.g. H′, Na+, Cl′, HCO′, K′ etc.). Alternatively the ion channel may respond to the binding of larger molecules derived from the targeted cell (for instance a growth factor, component of the extracellular matrix of mammalian cells or capsule polysaccharides of microorganisms). It is also possible to genetically engineer a peptide such that it will be responsive to any predetermined metabolic signal from a selected cell.

In certain embodiments, the peptide can be an integral protein of the lipid bilayer (i.e. the peptide spans the lipid bilayer). However it will be appreciated that the peptide may interact with the lipid bilayer in other ways (e.g. non-covalently attached to the outer lipid layer).

In some embodiments, the signal-generating element may be a polypeptide (e.g., a cytolytic peptide) which comprises an activatable structure, such as a chemical bond or constituent. The activatable structure, when activated, can enhance the capability of the polypeptide to modulate the permeability of a membrane. In some embodiments, the activatable structure may be a chemical, such as a phosphate group, which is responsive to pH. For example, the protonation of the phosphate group may activate the cytolytic activity of the signal-generating element. Such protonation may be mediated by the metabolic activity (e.g. production of acidic metabolites) of the target microorganisms. Several exemplary cytolytic peptides, GALA and LAGA, become activated as the pH drops from 7 to about 5.0 (Advanced Drug Delivery Reviews 56, (2004) 967-985). Conversely, another cytolytic peptide, KALA, is activated as the pH is raised from 5 to about 7.5. International Patent Application Number PCT/GB02/00033 discloses some peptides which are activated between 6.5 and 7.4, and other peptides which are triggered below 6.0, or 5.5.

In alternative embodiments, the activatable structure is activated by hydrolysis of a chemical bond, such as a phosphate ester. Hydrolysis of the bond may, for example, reduce the polarity (hydrophilicity) of the cytolytic peptide and/or may alter the isoelectric point of the peptide and thereby cause the polypeptide to modulate the permeability of a membrane. Such hydrolysis of a bond may be mediated by the metabolic activity (e.g. a phosphatase enzyme activity) of the target microorganism.

Chemical agents which may act as a signal-generating element include chemicals which can cause a latent signaling element to generate a detectable signal. For example, if the detectable signal is sequestered in a lipid vesicle, a chemical which can increase the permeability of the lipid vesicle (e.g., a detergent or surfactant) can serve as a signal-generating element. Additionally, a salt (e.g., NaCl) at an effective concentration to cause the plasmolysis of a lipid vesicle may serve as a signal-generating element.

### Linking Moiety

Linking moieties are molecules that may be used to join two or more other molecules (e.g. polypeptides). Linking moieties can be used to provide distance between the two linked molecules, thereby reducing the possibility of steric hindrance of binding sites or enzyme active sites. An example of one linking moiety includes 1-Ethyl-3-[3-dimethylaminopropyl]carbodiimide Hydrochloride, which can be used to couple a molecule with a carboxyl group to another molecule with an amine group.

In some embodiments, the linking moieties may comprise binding partners, such as biotin-binding polypeptide (e.g. avidin or streptavidin). Biotinylation kits are available from a number of commercial sources, such as Pierce Biochemical (Rockford, Ill.). Certain biotinylating agents include “spacer arms” comprising polyethylene oxide and/or polyethylene glycol. Hydrazide-modified streptavidin, for example, may be used to link streptavidin to a polypeptide (e.g., an antibody or receptor protein) for use as a biotin-binding partner.

### Methods of Detection

In one aspect, the present disclosure provides methods for the detection of target microorganisms in a non-growth dependent assay. As used herein, “non-growth dependent assay” refers to a test for target microorganisms that does not include a step wherein growth nutrients are provided and an incubation step is performed under conditions that are intended to cause the growth and multiplication of the target microorganisms. It should be noted that, although the non-growth dependent assays do not require a growth step as a component of the method, a non-growth dependent assay may be preceded by a growth step, which may improve the ability to detect relatively low numbers of target microorganisms.

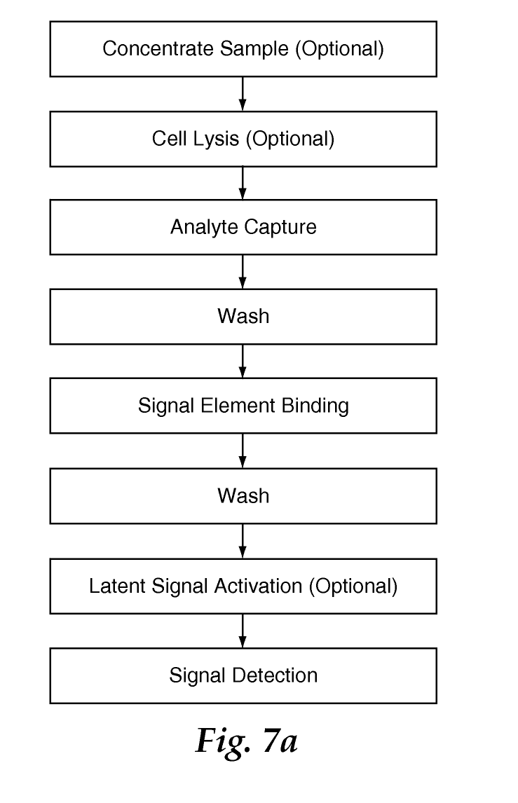

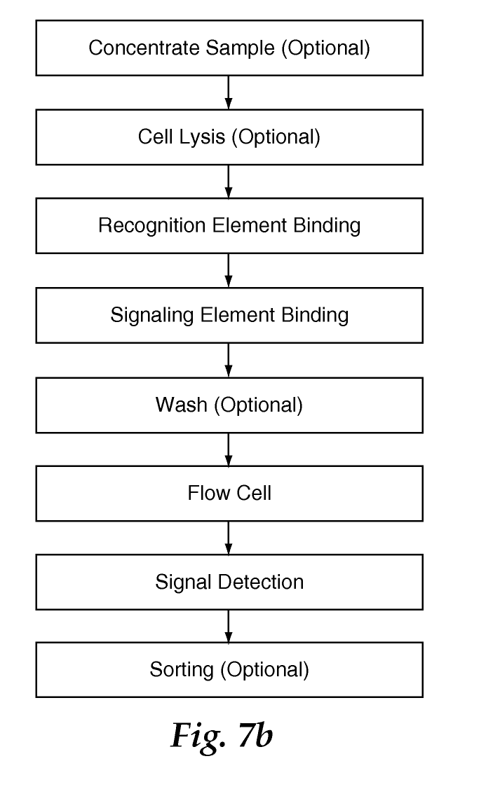

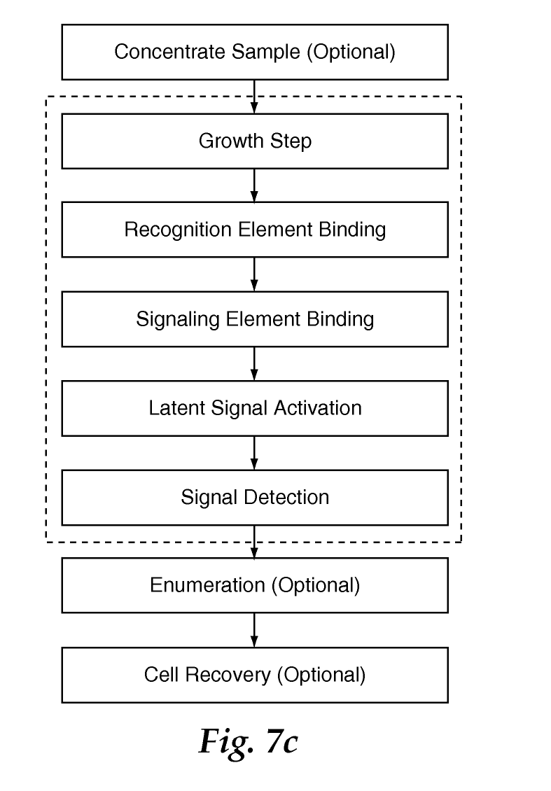

FIG. 7a shows a block diagram of an exemplary non-growth dependent assay. In the illustrated embodiment, the sample optionally may be concentrated (e.g., by filtration or centrifugation). The sample optionally may be treated with a lysing agent to enhance the interaction between the recognition element (e.g., an antibody) and its corresponding binding partner (e.g. a cell wall component such as zymosan). Suitable lysing agents may include chemical agents such as detergents and/or physical agents such as heat or ultrasonic vibration. After the concentration step and/or the lysis step, if either is desired, the target microorganism (or component thereof) is contacted with a capture agent attached to a support material (see FIG. 3) under conditions effective to capture one or more analytes characteristic of one or more fungal microorganisms, if present in the sample, to form one or more captured analytes. In some embodiments, the analytes are cell surface components of an intact microorganism. The captured analytes are contacted with recognition elements and their corresponding signaling elements under conditions effective to cause binding between the captured analytes, the recognition elements and signaling elements.

It should be noted also that the lysis step, the target capture step, and the step of binding the recognition and signaling elements may be conducted separately or in combinations of two or more of the individual steps. Following the binding steps, the support material can be washed to remove non-specifically bound and/or unbound signaling element. Following the wash step, the latent signaling element, if one is present in the assay, can be activated to produce a detectable signal. Activation can include, for example, the addition of an enzyme substrate to the mixture and/or the disruption of a lipid vesicle by chemical or physical means. The detectable signal can be detected to determine the presence of the target microorganism in the sample. Optionally, the detectable signal can be measured and the number of target microorganisms in the original sample can be estimated.

FIG. 7b shows a block diagram of an alternative exemplary non-growth dependent assay. In the illustrated embodiment, the sample optionally may be concentrated (e.g., by filtration or centrifugation). The sample optionally may be treated with a lysing agent to enhance the interaction between the recognition element and its corresponding binding partner (e.g. a cell wall component such as zymosan). Suitable lysing agents may include chemical agents such as detergents and/or physical agents such as heat or ultrasonic vibration. After the concentration step and/or the lysis step, if either one of those steps is included, the target microorganism (or component thereof) can be contacted with recognition elements and their corresponding signaling elements under conditions effective to cause binding between the target microorganism, if at least one is present, the recognition elements, and signaling elements. The mixture may be washed to remove signaling element that may be nonspecifically bound to the nontarget microorganisms, although the method including fluorescent particles (described below) shows that the wash step can be optional.

The mixture can be passed through a flow system, such as a flow cytometer, with a detector. Optionally, the sample may be contacted with a capture agent bound to a support material under conditions effective to cause binding between a target microorganism and the capture agent. In some embodiments, the support material may be a fluorescent particle which fluoresces at a first wavelength. The captured material can be contacted with a recognition element labeled with a signaling element (e.g., comprising a fluorescent dye which fluoresces at a second wavelength) under conditions effective to cause binding between the recognition element and a captured target microorganism (or component thereof), if one is present. The support material can then be passed through a flow system such as a flow cytometer. Thus, in some embodiments, the detection of a fluorescent particle in the flow cell which fluoresces at both the first and second wavelengths may indicate the presence of a target microorganism in the sample. Optionally, in the flow cell system, the particles may be sorted and the positive particles may be subjected to confirmatory testing such as polymerase chain reaction (PCR) testing or culture methods, for example.

In another aspect, the present disclosure provides methods for the detection of target microorganisms in a growth-dependent assay. Growth dependent assays include a step to increase the number of target microorganisms by providing an environment (e.g., nutrients, temperature) which enables the target microorganisms to undergo growth, metabolism, and/or cell division.

FIG. 7c shows a block diagram of an exemplary growth-dependent assay. In the illustrated embodiment, the sample optionally may be concentrated (e.g., by filtration or centrifugation) prior to the growth step. The sample can be placed into a culture device containing a nutrient medium. The nutrient medium and the incubation temperature can be chosen according to the growth requirements of the target microorganism. The nutrient medium can be contained in a variety of culture devices, such as petri dishes, rehydratable culture devices (e.g., film culture devices sold by 3M Company, St. Paul, Minn., under the trade name PETRIFILM), microtiter plates, flasks, tubes, and the like. Prior to the growth step or, optionally, after the growth step, the recognition and signaling elements can be added to the sample mixture under conditions effective to cause the binding of the recognition and signaling elements to the target microorganisms (or components thereof).

In this embodiment, a latent signaling element, which is activated by a metabolic activity of the target microorganisms, can be used. For example, the latent signaling element can include a signal-generating element (e.g., cytolytic peptides such as those described in International Patent Application Nos. PCT/GB98/03071 or PCT/GB02/00033). Such cytolytic peptides form pores in lipid vesicles when the pH of the environment is altered (e.g., by the metabolism of nutrients to acidic byproducts), thereby permitting the formation of pores. Alternatively, functional groups, such as phosphate groups, on the cytolytic peptides may be hydrolyzed, thereby permitting the formation of pores in the lipid vesicle (see FIG. 6 showing how pore formation can cause a detectable signal). Yeast and mold microorganisms are known to produce phosphatase enzymes which could be used in the activation of a cytolytic peptide. After activation of the latent signaling element, the signal can be detected.

In some embodiments involving nutrient growth medium, the individual target microorganisms from the sample may be spatially separated (e.g., on the surface of an agar plate) such that the location of individual colonies or microcolonies (after the growth and signal detection steps) may be observed. Embodiments including spatial separation of the target microorganisms from the original sample may include an optional enumeration step wherein the individual colonies or microcolonies are counted. A further optional step may include the recovery of target microorganisms (e.g., picking a colony) for subsequent archiving or additional testing (e.g., PCR analysis).

It should be recognized that some of the steps in the growth-dependent assay may be performed simultaneously. For example, the steps shown in the dashed box in FIG. 7c may be performed simultaneously, relying on the metabolic activity of the growing cells to activate the latent signal. When the indicated steps are performed simultaneously, it may be possible to monitor the culture device continuously or at various times to observe detectable signals. This process could provide for early detection of target microorganisms.

Preferably, in the above-described methods, providing contact between the sample, the immobilized antibodies, and the labeled recognition elements (e.g., antibodies) includes: contacting the sample with the immobilized antibodies under conditions effective to capture one or more analytes characteristic of a specific target microorganism, if present in the sample, to form one or more captured analytes; and contacting the one or more captured analytes, if present, with the labeled recognition elements under conditions effective to cause binding between the one or more captured analytes and the labeled recognition elements. Preferably, contacting the sample with the capture agents includes providing contact between the sample and each capture agent simultaneously. Preferably, contacting the one or more captured target microorganisms, if present, with the labeled recognition elements includes providing contact between the captured target microorganisms and each labeled recognition element simultaneously.

### EXAMPLES

### Example 1

### A Rabbit-Anti-Zymosan Antibody Recognizes Zymosan by an Indirect ELISA

### A. General Materials and Methods

Coating Buffer (0.1M Bicarbonate/Carbonate buffer, pH 9.6) was prepared as a 1× stock solution by mixing 3.03 g Na₂CO₃ and 6.0 g NaHCO₃ in 1000 mL distilled water. Phosphate-buffered saline (PBS, 155 mM NaCl in 10 mM phosphate buffer, pH 7.4) was prepared from a 10× stock solution obtained from Biosource (Rockville, Md.). BD Falcon Microtest™ 96-well enhanced surface ELISA plates were obtained from BD Biosciences (Bedford, Mass.). All procedures were performed at room temperature unless specified otherwise. All ELISA wash procedures included three sequential wash volumes of 200 microliters per well and all washes were done with PBS buffer. Horseradish peroxidase chromogenic substrate, 3,3′,5,5′-Tetramethylbenzidine (TMB), was obtained from Pierce Biotechnology (Rockford, Ill.). Sulfuric acid, 2M, was prepared from a stock concentrate (18.4M) obtained from Mallinckrodt Baker (Phillipsburg, N.J.). Bovine serum albumin (BSA) was used in this experiment as a 1% solution in PBS and obtained from Sigma-Aldrich (St. Louis, Mo.).

Antigens used in this experiment included Zymosan (Catalog #tlrl-zyn) from InvivoGen (San Diego, Calif.). Zymosan was reconstituted to a 1 mg/mL solution in water and stored at 4° C.

Antibodies used in this experiment included Rabbit-anti-Zymosan (Catalog #Z2850) from Molecular Probes (Carlsbad, Calif.) and Donkey-anti-Rabbit IgG, Horseradish-peroxidase(HRP)-conjugated (Catalog #NA934V) from Amersham Biosciences (Buckinghamshire, England).

### B. ELISA Conditions

Zymosan was used as a coating antigen in this assay. Zymosan was diluted from its refrigerated storage starting concentration to microwell coating concentrations (20 micrograms per well) in Coating Buffer. Two hundred microliters of the coating solution were added to the wells of the microwell plates. Plates were incubated on a rocking platform (Barnstead Lab-Line Maxi Rotator, Dubuque, Iowa) at a speed of 100 rpm at ambient temperature for 120 minutes. The coating solution was removed by washing with PBS prior to the blocking step.

The microtiter plate was blocked with two hundred microliters per well of 1% BSA diluted in PBS on a rocking platform at ambient temperature for 60 minutes. The blocking solution was removed by washing with PBS.

Four hundred microliters of the Rabbit-anti-Zymosan antibody (Molecular Probes #Z-2850) diluted to 2000 ng/mL in PBS was added to Row A. The rest of the wells contained 200 microliters of PBS. The Rows were diluted two-fold as follows: Two hundred microliters was taken from and Row A and diluted into Row B, then two hundred microliters from Row B was diluted into Row C, Row C was diluted into Row D, Row D was diluted into Row E, Row E was diluted into Row F, and Row F was diluted into Row G. Row H contained no Rabbit-anti-Zymosan as a blank. Two hundred microliters was discarded from Row G to maintain a consistent volume of two hundred microliters per well. The concentration of Rabbit-anti-Zymosan Antibody ranged from 2000 ng/mL to 31.25 ng/mL. The plate was incubated for 120 minutes on a rocking platform at ambient temperature. The plate was then washed with PBS.

Donkey-anti-Rabbit IgG, HRP-conjugated (Amersham #NA934V), was diluted in PBS by a factor of 1:625. Four hundred microliters of (the diluted?) Donkey-anti-Rabbit IgG, HRP-conjugated, was added to Columns 11 and 12. The rest of the wells contained two hundred microliters of PBS. To obtain duplicate samples, the Donkey-anti-Rabbit IgG, HRP-conjugated, was diluted two-fold across the plate. Two hundred microliters was taken from Column 11 and added to Column 9, then two hundred microliters from Column 9 was added to Column 7, then two hundred microliters from Column 7 was added to Column 5, then two hundred microliters from Column 5 was added to Column 3, then two hundred microliters from Column 3 was added to Column 1. Two hundred microliters was discarded from Column 1 to maintain a consistent volume of two hundred microliters per well. Likewise, two hundred microliters was taken from Column 12 and added to Column 10, then two hundred microliters from Column 10 was added to Column 8, then two hundred microliters from Column 8 was added to Column 6, then two hundred microliters from Column 6 was added to Column 4, then two hundred microliters from Column 4 was added to Column 2. Two hundred microliters was discarded from Column 2 to maintain a consistent volume of two hundred microliters per well. The Donkey-anti-Rabbit IgG, HRP-conjugated, dilution factors ranged from 1:625 to 1:20,000. The plate was incubated for 60 minutes on a rocking platform at ambient temperature. The plate was washed with PBS.

One hundred microliters of TMB substrate was added to each well. The plates were incubated at room temperature for 15 minutes to observe color development. The peroxidase reaction was stopped by adding one hundred microliters of 2M sulfuric acid to each well. The plate was placed in a plate reader, where the absorbance at 450-nm wavelength was read.

The antibody used in these experiments was a Rabbit-anti-Zymosan antibody. The secondary antibody used was an anti-Rabbit IgG, HRP-conjugated, that recognized all primary antibodies raised in rabbits. A titration curve was run to determine whether these antibodies recognized Zymosan. As shown in Table 2, the titration provided a linear response of the antibodies to the target Zymosan.

TABLE 2
ELISA results indicating that Rabbit-anti-Zymosan binds Zymosan in a linear “dose/response” manner.

| | Absorbance at 450 nm | | | | | | | | | | | | Biotinylated Rabbit- |
|---|---|---|---|---|---|---|---|---|---|---|---|---|---|
| | 1 | 2 | 3 | 4 | 5 | 6 | 7 | 8 | 9 | 10 | 11 | 12 | anti-Zymosan (ng/mL) |
| A | 0.023 | 0.025 | 0.06 | 0.076 | 0.144 | 0.142 | 0.269 | 0.262 | 0.388 | 0.388 | 0.584 | 0.51 | 2000 |
| B | 0.016 | 0.007 | 0.046 | 0.038 | 0.099 | 0.065 | 0.138 | 0.154 | 0.246 | 0.241 | 0.355 | 0.358 | 1000 |
| C | −0.001 | 0.001 | 0.017 | 0.013 | 0.034 | 0.025 | 0.073 | 0.073 | 0.125 | 0.122 | 0.189 | 0.204 | 500 |
| D | −0.006 | 0.001 | 0.011 | 0.011 | 0.017 | 0.009 | 0.035 | 0.036 | 0.056 | 0.057 | 0.094 | 0.139 | 250 |
| E | −0.002 | −0.008 | 0.001 | 0.007 | 0.005 | 0.004 | 0.012 | 0.02 | 0.032 | 0.024 | 0.057 | 0.079 | 125 |
| F | −0.001 | −0.009 | 0.001 | 0.001 | 0 | 0.008 | 0.007 | 0.002 | 0.016 | 0.004 | 0.022 | 0.082 | 62.5 |
| G | −0.008 | −0.008 | 0.001 | 0.003 | 0.002 | −0.007 | 0.011 | 0.003 | 0.003 | 0 | 0.022 | 0.001 | 31.25 |
| H | 0 | 0 | 0 | 0 | 0 | 0 | 0 | 0 | 0 | 0 | 0 | 0 | 0 |

| | | | | | |
|---|---|---|---|---|---|
| 20000 | 10000 | 5000 | 2500 | 1250 | 625 |
| R² = 0.9853 | R² = 0.9832 | R² = 0.9909 | R² = 0.9968 | R² = 0.9825 | R² = 0.977 |

HRP-Conjugated Donkey-anti-Rabbit IgG Dilution

The concentrations of the primary antibody are listed in nanograms/milliliter for each row. The HRP-conjugated Donkey-anti-Rabbit IgG is listed as a dilution factor for each column.

### Example 2

### Biotinylated Anti-Zymosan Recognizes Zymosan by a Biotin-Streptavidin ELISA

### A. General Materials and Methods

Coating Buffer, PBS, ELISA plates, incubation, and wash procedures were performed as described in Example 1. Alkaline phosphatase chromogenic substrate, p-nitrophenyl phosphate disodium salt (pNPP, Catalog #37621), was obtained from Pierce Biotechnology. Sodium hydroxide, 2N, was prepared from a stock concentration of 5M and obtained from Sigma-Aldrich. Bovine serum albumin (BSA) was used in this experiment as a 1% solution in PBS and obtained from Sigma-Aldrich. Immunopure Streptavidin, Alkaline Phosphatase (AP)-Conjugated, was obtained from Pierce Biotechnology.

Zymosan antigen was prepared and stored as described in Example 1.

Antibodies used in this experiment included Rabbit-anti-Zymosan (Catalog #Z2850) from Molecular Probes. Rabbit-anti-Zymosan was biotinylated using an EZ-Link Sulfo-NHS-Biotinylation Kit obtained from Pierce Biotechnology.

### B. ELISA Conditions

ELISA conditions were similar to those described in Example 1 with the exceptions that i) biotinylated rabbit-anti-zymosan antibody (Catalog #Z-2850, Molecular Probes) was used in place of the rabbit-anti-zymosan antibody, ii) alkaline phosphatase (AP)-conjugated streptavidin (Catalog #21324, Pierce Biotechnology) was used in place of HRP-conjugated donkey-anti-rabbit IgG, and iii) pNPP substrate was used as described below in place of the TMB substrate.

One hundred microliters of pNPP substrate was added to each well. The plates were incubated at room temperature for 15 minutes to observe color development. The phosphatase reaction was stopped by adding one hundred microliters of 2N sodium hydroxide to each well. The plate was placed in a plate reader, where the absorbance at 405-nm wavelength was read.

TABLE 3
ELISA results indicating that Biotinylated Rabbit-anti-Zymosan binds Zymosan in a linear manner.

| | Absorbance at 450 nm | | | | | | | | | | | | Biotinylated Rabbit- |
|---|---|---|---|---|---|---|---|---|---|---|---|---|---|
| | 1 | 2 | 3 | 4 | 5 | 6 | 7 | 8 | 9 | 10 | 11 | 12 | anti-Zymosan (ng/mL) |
| A | 0.028 | 0.038 | 0.051 | 0.046 | 0.082 | 0.098 | 0.070 | 0.064 | 0.152 | 0.099 | 0.174 | 0.124 | 2000 |
| B | 0.032 | 0.021 | 0.027 | 0.030 | 0.022 | 0.028 | 0.035 | 0.027 | 0.062 | 0.067 | 0.065 | 0.094 | 1000 |
| C | 0.011 | 0.004 | 0.013 | 0.013 | 0.012 | 0.015 | 0.008 | 0.008 | 0.020 | 0.027 | 0.113 | 0.016 | 500 |
| D | 0.011 | 0.001 | −0.004 | 0.004 | 0.000 | −0.011 | 0.032 | 0.015 | 0.044 | 0.030 | 0.056 | 0.005 | 250 |
| E | 0.011 | 0.007 | 0.015 | 0.016 | 0.004 | −0.004 | −0.021 | 0.002 | 0.035 | 0.012 | 0.009 | 0.013 | 125 |
| F | 0.004 | −0.001 | −0.011 | −0.005 | −0.007 | 0.017 | 0.004 | 0.018 | −0.022 | −0.011 | −0.025 | 0.006 | 62.5 |
| G | 0.001 | −0.002 | 0.004 | −0.003 | −0.007 | −0.002 | −0.007 | 0.013 | 0.012 | −0.019 | 0.004 | 0.010 | 31.25 |
| H | 0 | 0 | 0 | 0 | 0 | 0 | 0 | 0 | 0 | 0 | 0 | 0 | 0 |

| | | | | | |
|---|---|---|---|---|---|
| 20000 | 10000 | 5000 | 2500 | 1250 | 625 |
| R² = 0.8888 | R² = 0.8685 | R² = 0.8456 | R² = 0.9415 | R² = 0.9102 | R² = 0.9479 |

Alkaline Phosphatase-Conjugated Streptavidin Dilution

The concentrations of the primary antibody are listed in nanograms/milliliter for each row. The AP-conjugated Streptavidin is listed as a dilution factor for each column.

The liposomes used in Examples 3 and 5 have biotinylated phospholipids, which means that antibodies were conjugated to these liposomes using biotin-streptavidin chemistry. This experiment was performed to optimize the conditions for using biotin-streptavidin chemistry to conjugate antibodies to liposomes. In this experiment, the anti-Zymosan antibody was biotinylated, and detection of the antibody binding Zymosan was done using an AP-conjugated Streptavidin. The titration data indicated a linear response of the biotinylated antibody and Streptavidin to Zymosan (Table 3).

### Example 3

### Liposomes Recognize Zymosan through Anti-Zymosan Antibody Interactions

### A. General Materials and Methods

Coating Buffer, PBS, ELISA plates, incubation, and wash procedures were performed as described in Example 1. Bovine serum albumin (BSA) was used in this experiment as a 1% solution in PBS and obtained from Sigma-Aldrich. ImmunoPure Streptavidin was obtained from Pierce Biotechnology.

A solution of 0.25% TRITON X-100 in water was used to lyse the liposomes. Horseradish peroxidase chromogenic substrate, 3,3′,5,5′-Tetramethylbenzidine (TMB), was obtained from Pierce Biotechnology. Sulfuric acid, 2M, was prepared from a stock concentrate, as described in Example 1.

Zymosan antigen was prepared and stored as described in Example 1.

Biotinylated rabbit-anti-zymosan antibody was prepared as described in Example 2.

### B. HRP Liposome Synthesis

1.25 ml of a 20 mg/ml CHCl₃ solution of 1,2-dipalmitoyl-sn-glycero-3-phosphocholine (DPPC) (Avanti Polar Lipids, Alabaster, Ala.) was measured into a flask. 5 μl of a 50 mg/ml CHCl₃ solution of 1,2-Dipalmitoyl-sn-Glycero-3-Phosphoethanolamine-N-(Biotinyl) (Sodium Salt) (Avanti Polar Lipids, Alabaster, Ala.) was then added. The combined solution was then dried on a rotary evaporator and placed under vacuum to remove excess solvent.

The dried lipid film was then hydrated with 1 ml of a 5 mg/ml solution of horseradish peroxidase (HRP, obtained from Sigma Aldrich) in 10 mmol N-Tris(hydroxymethyl)methyl]-2-aminoethanesulfonic acid (TES) the pH of which was adjusted to 6.5 with 1 N HCl.

This solution was then placed in an ultrasonic bath (Bransonic 2510, Branson Ultrasonic Corp., Danbury, Conn.) and sonicated for one hour at 50° C.

Following sonication the liposome solution was alternately frozen in a dry ice/acetone bath and then thawed in warm water five times. The cloudy solution was first extruded fifteen times through a 400 nm polycarbonate membrane (Avanti Polar Lipids) at 50°-55° C., and then through a 200 nm membrane at the same temperature.

Excess unencapsulated HRP was removed by column chromatography, first via a Q-XL anion exchange column (GE Healthcare, Piscataway, N.J.) followed by a Sephacryl S-300 size exclusion column (GE Healthcare), in each case being eluted with 10 mM TES. Lipid concentration was assessed with a Phospholipid C test (Wako Chemicals, Richmond Va.) at 4.13 mg/ml and the z-average particle determined to be 126 nm by dynamic light scattering (Malvern Zetasizer Nano ZS, Malvern Instruments Ltd, Worcestershire, UK).

### C. ELISA Conditions

Zymosan was used as a coating antigen in this assay. Zymosan was diluted from its refrigerated storage starting concentration to microwell coating concentrations (20 micrograms per well) in Coating Buffer. Two hundred microliters of the coating solutions were added to the wells of the microwell plates. Plates were incubated on a rocking platform at ambient temperature for 120 minutes. The coating solution was removed by washing with PBS prior to the blocking step.

The microtiter plate was blocked with two hundred microliters per well of 1% BSA diluted in PBS on a rocking platform at ambient temperature for 60 minutes. The blocking solution was removed by washing with PBS.

Two hundred microliters of the Biotinylated Rabbit-anti-Zymosan antibody (Molecular Probes #Z-2850) diluted to 2000 ng/mL in PBS were added to Rows A, B, E, and F. Rows C, D, G, and H contained two hundred microliters of PBS. The plate was incubated for 120 minutes on a rocking platform at ambient temperature. The plate was then washed with PBS.

Two hundred microliters of ImmunoPure Streptavidin (Pierce Biotechnology #21122) diluted to 5000 ng/mL in PBS was added to Rows A, B, C, and D. Rows E, F, G, and H contained two hundred microliters of PBS. The plate was incubated for 60 minutes on a rocking platform at ambient temperature. The plate was then washed with PBS.

The biotinylated liposomes were diluted in PBS by a factor of 1:625. Four hundred microliters of liposomes were added to all the wells in Column 6. The rest of the wells contained two hundred microliters of PBS. The liposomes were diluted two-fold across the plate. Two hundred microliters was taken from Column 6 and added to Column 5, then two hundred microliters from Column 5 was added to Column 4, then two hundred microliters from Column 4 was added to Column 3, then two hundred microliters from Column 3 was added to Column 2, then two hundred microliters from Column 2 was added to Column 1. Two hundred microliters was discarded from Column 1 to maintain a consistent volume of two hundred microliters per well. The liposome dilution factors ranged from 1:625 to 1:20,000. The plate was incubated for 60 minutes on a rocking platform at ambient temperature. The plate was washed with PBS.

Twenty five microliters of 0.25% Triton X-100 were added to each well to lyse the liposomes. Immediately after addition of the Triton X-100, One hundred microliters of TMB substrate was added to each well. The plates were incubated at room temperature for 15 minutes to observe color development. The peroxidase reaction was stopped by adding one hundred microliters of 2M sulfuric acid to each well. The plate was placed in a plate reader, where the absorbance at 450-nm wavelength was read.

In Table 4, results indicated that liposomes recognized Zymosan through antibody interactions. Liposomes containing biotinylated phospholipids were conjugated to biotinylated anti-Zymosan antibodies through a Streptavidin linker. Controls shown on the graph included omission of the anti-Zymosan antibody, omission of Streptavidin, or omission of both the antibody and Streptavidin.

TABLE 4
ELISA results indicating that liposomes recognize Zymosan through antibody-mediated interactions.

| | Absorbance at 450 nm | | | | | | |
|---|---|---|---|---|---|---|---|
| | 1 | 2 | 3 | 4 | 5 | 6 | |
| A | 0.183 | 0.302 | 0.546 | 0.794 | 1.014 | 1.143 | Biotinylated Rabbit anti-Zymosan + Streptavidin + |
| B | 0.194 | 0.325 | 0.531 | 0.749 | 1.052 | 1.127 | Biotinylated HRP-containing liposomes |
| C | 0.069 | 0.071 | 0.081 | 0.081 | 0.127 | 0.294 | Streptavidin + |
| D | 0.069 | 0.072 | 0.075 | 0.076 | 0.149 | 0.287 | Biotinylated HRP-containing liposomes |
| E | 0.061 | 0.063 | 0.067 | 0.077 | 0.121 | 0.250 | Biotinylated Rabbit anti-Zymosan + Biotinylated |
| F | 0.064 | 0.062 | 0.067 | 0.073 | 0.119 | 0.296 | HRP-containing liposomes |
| G | 0.061 | 0.066 | 0.070 | 0.077 | 0.120 | 0.323 | Biotinylated HRP-containing liposomes |
| H | 0.061 | 0.063 | 0.069 | 0.080 | 0.136 | 0.318 | |
| | 1:20000 | 1:10000 | 1:5000 | 1:2500 | 1:1250 | 1:625 | |

Biotinylated liposome dilution factor

The biotinylated liposomes are listed as a dilution factor for each column.

### Example 4

### Biotinylated Anti-Zymosan Binds Zymosan in Yeast and Mold Whole Cell Lysates by a Biotin-Streptavidin ELISA

### A. General Materials and Methods

Coating Buffer, PBS, ELISA plates, incubation, and wash procedures were performed as described in Example 1. Horseradish peroxidase chromogenic substrate, 3,3′,5,5′-Tetramethylbenzidine (TMB), was obtained from Pierce Biotechnology. Sulfuric acid, 2M, was prepared from a stock concentration of 18.4M and obtained from Mallinckrodt Baker (Phillipsburg, N.J.). Bovine serum albumin (BSA) was used in this experiment as a 1% solution in PBS and obtained from Sigma-Aldrich. Immunopure Streptavidin, Horseradish Peroxidase (HRP)-Conjugated, was obtained from Pierce Biotechnology.

Zymosan antigen was prepared and stored as described in Example 1.

Yeasts used in this experiment included Saccharomyces cerevisiae (ATCC #24297), Saccharomyces cerevisiae (ATCC #201390), and Candida albicans (ATCC #10231). Molds used in this experiment included Aspergillus flavus (ATCC #9643), Aspergillus niger (ATCC #16404), Cladosporium herbarum (ATCC #76226), and Penicillium funiculosum (ATCC #11797). Yeasts were cultured on Difco™ Sabouraud Agar obtained from BD Biosciences (San Jose, Calif.). Molds were cultured on Difco™ Potato Dextrose Agar obtained from BD Biosciences. C. albicans cultures were grown at 37° C. All other cultures were grown at 28° C.

Antibodies used in this experiment included Rabbit-anti-Zymosan (Catalog #Z2850) from Molecular Probes as a capture antibody. Rabbit-anti-Zymosan was biotinylated using an EZ-Link Sulfo-NHS-Biotinylation Kit obtained from Pierce Biotechnology and used as a primary antibody for detecting Zymosan in yeast and mold whole cell lysates.

### B. Yeast and Mold Whole Cell Lysate Conditions

Yeasts used in this experiment included Saccharomyces cerevisiae (ATCC #24297), Saccharomyces cerevisiae (ATCC #201390), and Candida albicans (ATCC #10231). Molds used in this experiment included Aspergillus flavus (ATCC #9643), Aspergillus niger (ATCC #16404), Cladosporium herbarum (ATCC #76226), and Penicillium funiculosum (ATCC #11797). Yeasts were cultured on Difco™ Sabouraud Agar obtained from BD Biosciences for two days before harvest. Molds were cultured on Difco™ Potato Dextrose Agar obtained from BD Biosciences for five days before harvest. Lawns were harvested by addition of RIPA buffer (10 mM Na₂HPO₄, pH 7, 150 mM NaCl, 0.1% SDS, 1% NP-40, 1% Sodium Deoxycholate, 2 mM EDTA). All chemicals for RIPA buffer were obtained from Sigma-Aldrich. After addition of RIPA buffer, the colonies were scraped from the plate using a spatula and transferred to 1.5 mL microcentrifuge tubes. Molds were filtered through glass wool to remove spores. The cells were sonicated for one minute at 50 Hertz using a Micro Ultrasonic Cell Disrupter obtained from Kimble/Kontes (Vineland, N.J.), and the lysates were then spun for 10 minutes at 13,000 rpm at 4° C. to pellet insoluble material. The supernatants were transferred to fresh tubes, and total protein concentration was determined using a BCA™ Protein Assay Kit obtained from Pierce Biotechnology. Twenty micrograms of each whole cell lysate was used for analysis.

### C. ELISA Conditions

Rabbit-anti-Zymosan was used as a capture antigen in this assay. Rabbit-anti-Zymosan was diluted from its frozen storage starting concentration to microwell coating concentrations (20 micrograms per well) in Coating Buffer. Two hundred microliters of the coating solutions were added to the wells of the microwell plates. Plates were incubated on a rocking platform at ambient temperature for 120 minutes. The coating solution was removed by washing with PBS prior to the blocking step.

The microtiter plate was blocked with two hundred microliters per well of 1% BSA diluted in PBS on a rocking platform at ambient temperature for 60 minutes. The blocking solution was removed by washing with PBS.

Twenty micrograms of whole cell lysates from Saccharomyces cerevisiae (ATCC #24297), Saccharomyces cerevisiae (ATCC #201390), Candida albicans (ATCC #10231), Aspergillus flavus (ATCC #9643), Aspergillus niger (ATCC #16404), Cladosporium herbarum (ATCC #76226), and Penicillium funiculosum (ATCC #11797) were diluted to a total volume of two hundred microliters in PBS. Zymosan was diluted from its refrigerated storage starting concentration to 20 micrograms per well in two hundred microliters of PBS. Zymosan was added to Rows A, B, and C of Column 1, Saccharomyces cerevisiae (ATCC #24297) was added to the corresponding row of Column 2, Saccharomyces cerevisiae (ATCC #201390) to Column 3, Candida albicans (ATCC #10231) to Column 4, Aspergillus flavus (ATCC #9643) to Column 5, Aspergillus niger (ATCC #16404) to Column 6, Cladosporium herbarum (ATCC #76226) to Column 7, and Penicillium funiculosum (ATCC #11797) to Column 8. Column 12 contained two hundred microliters of PBS only as a blank. Plates were incubated on a rocking platform at ambient temperature for 60 minutes. The plate was washed with PBS.

Two hundred microliters of the Biotinylated Rabbit-anti-Zymosan antibody (Molecular Probes #Z-2850) diluted to 5000 ng/mL in PBS was added to Row A. Two hundred microliters of the Biotinylated Rabbit-anti-Zymosan antibody diluted to 2500 ng/mL in PBS was added to Row B. Two hundred microliters of the Biotinylated Rabbit-anti-Zymosan antibody diluted to 1250 ng/mL in PBS was added to Row C. The plate was incubated for 120 minutes on a rocking platform at ambient temperature. The plate was washed with PBS.

Horseradish Peroxidase (HRP)-conjugated Streptavidin, was diluted in PBS to a working concentration of 100 ng/mL. Two hundred microliters of Streptavidin, HRP-conjugated, was added to each well. The plate was incubated for 60 minutes on a rocking platform at ambient temperature. The plate was washed with PBS.

One hundred microliters of TMB substrate was added to each well. The plates were incubated at room temperature for 15 minutes to observe color development. The peroxidase reaction was stopped by adding one hundred microliters of 2M sulfuric acid to each well. The plate was placed in a plate reader, where the absorbance at 450-nm wavelength was read.

Results, shown in Table 5, indicated that the biotinylated Rabbit-anti-Zymosan antibody recognized Zymosan in whole cell lysates from both yeasts and molds. Purified Zymosan was used as a control.

TABLE 5
ELISA results indicating that Biotinylated Rabbit-anti-Zymosan
antibodies bind Zymosan in yeast and mold whole cell lysates.

| | Biotinylated Rabbit Anti-Zymosan | | |
|---|---|---|---|
| Whole cell lysate | 5.0 ug | 2.5 ug | 1.25 ug |
| Purified Zymosan (InvivoGen | 0.045 | 0.022 | 0.010 |
| tlrl-zyn) | | | |
| S. cerevisiae ATCC #24297 | 0.167 | 0.211 | 0.098 |
| S. cerevisiae ATCC #201390 | 0.152 | 0.154 | 0.076 |
| C. albicans ATCC #10231 | 0.167 | 0.141 | 0.079 |
| A. flavus ATCC #9643 | 0.224 | 0.173 | 0.066 |
| A. niger ATCC #16404 | 0.485 | 0.319 | 0.154 |
| C. herbarum ATCC #76226 | 0.133 | 0.058 | 0.034 |
| P. funiculosum ATCC #11797 | 0.251 | 0.237 | 0.135 |

Absorbance 450 nm
The concentrations of the primary antibody are listed in nanograms/milliliter for each Column.

### Example 5

### Liposomes Recognize Zymosan in Yeast and Mold Whole Cell Lysates Through Antibody Interactions

### A. General Materials and Methods

Coating Buffer, PBS, ELISA plates, incubation, and wash procedures were performed as described in Example 1. Bovine serum albumin (BSA) was used in this experiment as a 1% solution in PBS and obtained from Sigma-Aldrich. ImmunoPure Streptavidin was obtained from Pierce Biotechnology.

The liposomes used contained DPPC/Biotin with 5 mg/mL HRP in 10 mM TES, pH 6.5. A solution of 0.25% Triton X-100 in water was used to lyse the liposomes. Horseradish peroxidase chromogenic substrate, 3,3′,5,5′-Tetramethylbenzidine (TMB), was obtained from Pierce Biotechnology. Sulfuric acid, 2M, was prepared from a stock concentration of 18.4M and obtained from Mallinckrodt Baker (Phillipsburg, N.J.).

Antigens used in this experiment included Zymosan (Catalog #tlrl-zyn) from InvivoGen. Zymosan was reconstituted to a 1 mg/mL solution in water and stored at 4° C.

Yeasts and molds used in this experiment were prepared as described in Example 4. Antibodies used in this experiment were prepared as described in Example 4.

### B. Yeast and Mold Whole Cell Lysate Conditions

Yeast and mold whole cell lysate conditions and procedures were as described in Example 4.

### C. HRP Liposome Synthesis

Liposomes were synthesized as described in Example 3.

### D. ELISA Conditions

Plates were coated with rabbit-anti-zymosan and were blocked with BSA as described in Example 4.

Whole cell lysates and zymosan were added to the wells of the plates and the plates were incubated and washed as described in Example 4.

Two hundred microliters of the Biotinylated Rabbit-anti-Zymosan antibody (Molecular Probes #Z-2850) diluted to 5000 ng/mL in PBS was added to each well. The plate was incubated for 120 minutes on a rocking platform at ambient temperature. The plate was washed with PBS.

Two hundred microliters of ImmunoPure Streptavidin (Pierce Biotechnology #21122) diluted to 5000 ng/mL in PBS was to each well. The plate was incubated for 60 minutes on a rocking platform at ambient temperature. The plate was then washed with PBS.

The biotinylated liposomes were diluted in PBS by a factor of 1:625. Four hundred microliters of liposomes were added to all the wells in Row A. The rest of the rows contained two hundred microliters of PBS. The liposomes were diluted two-fold across the plate. Two hundred microliters was taken from Row A and added to Row B, then two hundred microliters from Row B was added to Row C, then two hundred microliters from Row C was added to Row D, then two hundred microliters from Row D was added to Row E, then two hundred microliters from Row E was added to Row F, then two hundred microliters from Row F was added to Row G, then two hundred microliters from Row G was added to Row H. Two hundred microliters was discarded from Row H to maintain a consistent volume of two hundred microliters per well. The liposome dilution factors ranged from 1:625 to 1:40,000. The plate was incubated for 60 minutes on a rocking platform at ambient temperature. The plate was washed with PBS.

Twenty five microliters of 0.25% Triton X-100 were added to each well to lyse the liposomes. Immediately after addition of the Triton X-100, One hundred microliters of TMB substrate was added to each well. The plates were incubated at room temperature for 15 minutes to observe color development. The peroxidase reaction was stopped by adding one hundred microliters of 2M sulfuric acid to each well. The plate was placed in a plate reader, where the absorbance at 450-nm wavelength was read.

Results, shown in Table 6, indicated that liposomes recognized Zymosan in whole cell lysates from yeasts and molds through antibody interactions.

TABLE 6
ELISA results indicating that liposomes bind Zymosan in yeast and
mold whole cell lysates through antibody-mediated interactions.

| | Absorbance 450 nm | | | | | | | | Liposome |
|---|---|---|---|---|---|---|---|---|---|
| | 1 | 2 | 3 | 4 | 5 | 6 | 7 | 8 | Dilution |
| A | 0.062 | 0.215 | 0.467 | 0.858 | 0.576 | 0.937 | 0.564 | 0.679 | 625 |
| B | 0.043 | 0.435 | 0.501 | 0.911 | 0.565 | 0.882 | 0.587 | 0.697 | 1250 |
| C | 0.060 | 0.199 | 0.267 | 0.215 | 0.361 | 0.512 | 0.353 | 0.541 | 2500 |
| D | 0.092 | 0.196 | 0.211 | 0.162 | 0.260 | 0.329 | 0.251 | 0.389 | 5000 |
| E | 0.045 | 0.114 | 0.101 | 0.089 | 0.162 | 0.265 | 0.150 | 0.237 | 10000 |
| F | 0.035 | 0.073 | 0.070 | 0.059 | 0.088 | 0.143 | 0.080 | 0.139 | 20000 |
| G | 0.001 | 0.029 | 0.024 | 0.017 | 0.046 | 0.076 | 0.033 | 0.065 | 40000 |
| H | 0.007 | 0.022 | 0.026 | 0.018 | 0.025 | 0.042 | 0.021 | 0.029 | 80000 |
| Whole | Purified | S. cerevisiae | S. cerevisiae | C. albicans | A. flavus | A. niger | C. herbarum | P. funiculosum | |
| cell | Zymosan | ATCC | ATCC | ATCC | ATCC | ATCC | ATCC | ATCC | |
| lysate | (InvivoGen) | #24297 | #201390 | #10231 | #9643 | #16404 | #76226 | #11797 | |

The biotinylated liposomes are listed as a dilution factor for each row.

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

1. A method for detecting a target microorganism or a component thereof, comprising:
providing a recognition element that selectively binds to zymosan;
contacting the recognition element with a sample suspected of containing the target microorganism; and
detecting the target microorganism.

2. A method for detecting a target microorganism or a component thereof, comprising:
providing a recognition element that selectively binds to zymosan;
providing a signaling element which generates a detectable signal, wherein the signaling element comprises a linking moiety;
contacting the recognition element and the signaling element with a sample suspected of containing the target microorganism; and
detecting the detectable signal.

3. A method for detecting a target microorganism or a component thereof, comprising:
providing a recognition element that selectively binds to zymosan;
providing a signaling element which generates a detectable signal, wherein the recognition element is linked to the signaling element;
contacting the recognition element and the signaling element with a sample suspected of containing the target microorganism; and
detecting the detectable signal.

4. A method for detecting a target microorganism or a component thereof, comprising:
providing a lipid vesicle comprising a lipid bilayer and a signaling element which generates a detectable signal;
providing a recognition element that selectively binds to a cell wall component;
contacting the lipid vesicle and the recognition element with a sample suspected of containing the target microorganism under conditions effective to cause the binding of the lipid vesicle to the target microorganism, if one is present; and
detecting the detectable signal.

5. The method of claim 4, wherein the lipid bilayer comprises at least one biotin molecule.

6. The method of claim 4, wherein the recognition element comprises at least one biotin molecule.

7. The method of claim 4, further comprising providing a biotin-binding molecule.

8. The method of claim 1, further comprising steps of i) providing a nutrient medium in a culture device and ii) incubating the sample under conditions to allow for at least one cell division.

9. The method of claim 1, the method further comprising a step of removing a viable microorganism from the culture device.

10. The method of claim 1, further comprising the step of lysing a microorganism in the sample.

11. The method of claim 1, further comprising the steps of i) providing a capture reagent and ii) capturing the target microorganism or a component thereof.

12. The method of claim 2, wherein the signaling element comprises a lipid vesicle, a magnetic particle, a polymeric particle, or a gold particle.

13. The method of claim 2, wherein the signaling element comprises a polypeptide.

14. The method of claim 2, wherein the signaling element comprises a polynucleotide.

15. The method of claim 2, wherein the signaling element comprises a latent signaling component which is converted to a detectable signal.

16. The method of claim 15, further comprising the steps of i) providing a signal-generating element and ii) contacting the signal-generating element with the signaling element to convert the latent signaling component to a detectable signal.

17. The method of claim 16 wherein the signal-generating element is an activatable signal-generating element and further comprising the step of activating the activatable signal-generating element.

18. The method of claim 17, wherein the activatable signal-generating element comprises a polypeptide.

19. The method of claim 18 wherein the polypeptide comprises an activatable structure which, when activated, enhances the capability of the polypeptide to modulate the permeability of a membrane.

20. The method of claim 19, wherein the activatable structure comprises a hydrolysable bond.

21. The method of claim 18 wherein the polypeptide is activated by a pH change.

22. The method of claim 1, further comprising the step of enumerating the target microorganisms in the sample.

23. The method of claim 1, wherein the detectable signal can be detected optically.

24. The method of claim 23 wherein detecting optically comprises colorimetric detection, fluorometric detection, or lumimetric detection.

25. The method of claim 23, wherein detecting optically comprises detecting with an instrument.

26. A composition for detecting a target microorganism, comprising:
a recognition element that selectively binds to zymosan; and
a signaling element comprising a particle which generates a detectable signal.

27. The signaling element of claim 26, wherein the particle comprises a lipid vesicle.

28. The signaling element of claim 26, wherein the particle comprises a fluorescent molecule.

29. The signaling element of claim 26, wherein the particle comprises a magnetic particle.

30. The signaling element of claim 26, wherein the particle comprises a polymeric particle.

31. The signaling element of claim 26, wherein the particle comprises a gold particle.

32. The signaling element of claim 26, wherein the particle comprises a polypeptide.

33. The composition of claim 26, further comprising a linking moiety.

34. The composition of claim 33 wherein the recognition element is linked to the signaling element.

35. The composition of claim 26, wherein the signaling element comprises a latent signaling element which is converted to a detectable signal.

36. The signaling element of claim 35 wherein the latent signaling element comprises an enzyme substrate.

37. The signaling element of any of claim 35, wherein the signaling element comprises a polypeptide.

38. The polypeptide of claim 37, wherein the polypeptide comprises an enzyme activity.

39. The composition of claim 35, further comprising a signal-generating element wherein the signal-generating element converts the latent signaling element to a detectable signal.

40. The composition of claim 39 wherein the signal-generating element is activatable.

41. The composition of claim 40, wherein the activatable signal-generating element comprises a polypeptide.

42. The polypeptide of claim 41, wherein the polypeptide comprises an activatable structure which, when activated, enhances the capability of the polypeptide to modulate the permeability of a membrane.

43. The composition of claim 41, wherein the activatable structure comprises a hydrolysable bond.

44. The composition of claim 43 wherein the hydrolysable bond is an ester bond or an amide bond.

45. The composition of claim 41, wherein the signal-generating element is activated by a pH change.

46. The composition of claim 26, further comprising a capture agent.

47. The composition of claim 46, wherein the capture agent is attached to a solid support.

48. The composition of claim 47, wherein the solid support is selected from the group consisting of a plastic film, a plastic article, a metal film, a particle, a membrane, a hydrogel, a cellulosic composition, a nonwoven, a fiber, a microchannel.

49. The composition of claim 46, wherein the capture agent or the recognition element selectively binds to zymosan.

50. The composition of claim 46, wherein the capture agent and the recognition element selectively bind to zymosan.

51. The composition of claim 26, wherein recognition element or the capture agent comprises a polypeptide which includes a zymosan binding domain.

52. The composition of claim 51, wherein the polypeptide comprises an antibody, or antigen-binding fragment derived thereof, which selectively binds to zymosan.

53. The antigen-binding fragment thereof of claim 52, wherein the antigen-binding fragment thereof is selected from the group consisting of a Fab fragment, a Fab′ fragment, a F(ab)₂ fragment, a single-chain Fv, and a Fv fragment.

54. The polypeptide of claim 51, wherein the polypeptide comprises a receptor.

55. The receptor of claim 54 wherein the receptor comprises a carbohydrate-recognition domain from Dectin-1.

56. The polypeptide of claim 51, wherein the polypeptide binds to the same epitope of zymosan which is recognized by the carbohydrate-recognition domain of Dectin-1.
